(19) United States
(12) Patent Application Publication
(10) Pub. No.: US 2014/0378903 A1
(43) Pub. Date: Dec. 25, 2014

(54) MANUALLY ACTUATED INFUSION DEVICE AND DOSE COUNTER

(71) Applicant: ANIMAS CORPORATION, WEST CHESTER, PA (US)

(72) Inventor: John QUINLAN, Spring City, PA (US)

(73) Assignee: ANIMAS CORPORATION, WEST CHESTER, PA (US)

(21) Appl. No.: 14/289,930

(22) Filed: May 29, 2014

Related U.S. Application Data
(60) Provisional application No. 61/837,697, filed on Jun. 21, 2013.

(51) Int. Cl. A61M 5/142; A61M 5/168

(52) U.S. Cl. CPC A61M 5/14216; A61M 5/14248; A61M 5/16881; A61M 2005/14208; A61M 2202/07; A61M 2205/35
USPC 604/152; 604/151

(57) ABSTRACT

An infusion device for delivering discrete boluses of medication to a patient, using a mechanically actuated piston. The infusion pump employs a near-field communication system to convey the occurrence of an actuation, the amount of medicament remaining in the pump, and other information to a nearby near-field receiver device. The disclosed drive system and nearer-field communications system provides an infusion device capable of accurately delivering medication and providing a means for tracking and logging data while eliminating the need for a power source within the device, thereby minimizing weight and size.

### RELATED APPLICATIONS

This application claims priority to U.S. Ser. No. 61/837,697 filed Jun. 21, 2013, which application is incorporated herein by reference.

### FIELD OF THE INVENTION

The present invention relates to infusion devices and more particularly to such devices that enable liquid medicaments to be conveniently and safely self-administered by a patient.

### BACKGROUND OF THE INVENTION

Tight control over the delivery of insulin in both type I diabetes (usually juvenile onset) and type II diabetes (usually late adult onset), has been shown to improve the quality of life as well as the general health of these patients. Insulin delivery has been dominated by subcutaneous injections of both long acting insulin to cover the basal needs of the patient and by short acting insulin to compensate for meals and snacks. Recently, the development of electronic, external insulin infusion pumps has allowed the continuous infusion of fast acting insulin for the maintenance of the basal needs as well as the compensatory doses (boluses) for meals and snacks. These infusion systems have shown to improve control of blood glucose levels. However, they suffer the drawbacks of size, cost, and complexity. For example, these pumps are electronically controlled and must be programmed to supply the desired amounts of basal and bolus insulin. This prevents many patients from accepting this technology over the standard subcutaneous injections.

Hence, there is a need in the art for a convenient form of insulin treatment which does not require significant programming or technical skills to implement to service both basal and bolus needs. Preferably, such a treatment would be carried out by an infusion device that is simple to use and mechanically driven negating the need for batteries and the like. It would also be preferable if the infusion device could be directly attached to the body and not require any electronics to program the delivery rates. The insulin is preferably delivered through a small, thin-walled tubing (cannula) through the skin into the subcutaneous tissue similar to technologies in the prior art.

While the idea of such a simple insulin delivery device is compelling, many obstacles must be overcome before such a device may become a practical realty. One problem resides in insulin supply. Patients vary greatly on the amount of insulin such a device must carry to provide treatment over a fixed time period of, for example, three days. This is one environment where one size does not fit all. Still further, such devices must be wearable with safety and not subject to possible accidental dosing. Still further, such devices must be capable of delivering an accurately controlled volume of medicament with reliability. Finally, a device that provides means for tracking the number of doses of medication delivered is highly desirable to permit a patient or healthcare provider to ensure that the correct amount of medication is administered over a given period of time. While it is preferred that these devices include all of the forgoing features, it would be further preferred if the cost of manufacturing such a device would be economical enough so as to render the device disposable after use. As will be seen subsequently, the devices and methods described herein address these and other issues.

### BRIEF DESCRIPTION OF THE DRAWINGS

The features of the present invention which are believed to be novel are set forth with particularity in the appended claims. The invention, together with further features and advantages thereof, may best be understood by making reference to the following description taken in conjunction with the accompanying drawings, in the several figures of which like reference numerals identify identical elements, and wherein:

FIG. 1 is a perspective view of a first infusion device embodying certain aspects of the present invention.

FIG. 2 is a schematic representation of the valves and pump of the device of FIG. 1.

FIG. 3 is an exploded perspective view of the device of FIG. 1.

FIG. 4 shows in perspective view a near-field antenna electrically coupled to an encoder system according to an aspect of the present invention.

FIG. 5 illustrates in perspective view a geared encoder disc with a ratchet and pawl system for rotating the disc in discrete increments.

FIG. 6 illustrates an embodiment of the infusion device of the present invention, in perspective view, including the encoder system of FIG. 5.

### DETAILED DESCRIPTION OF THE INVENTION

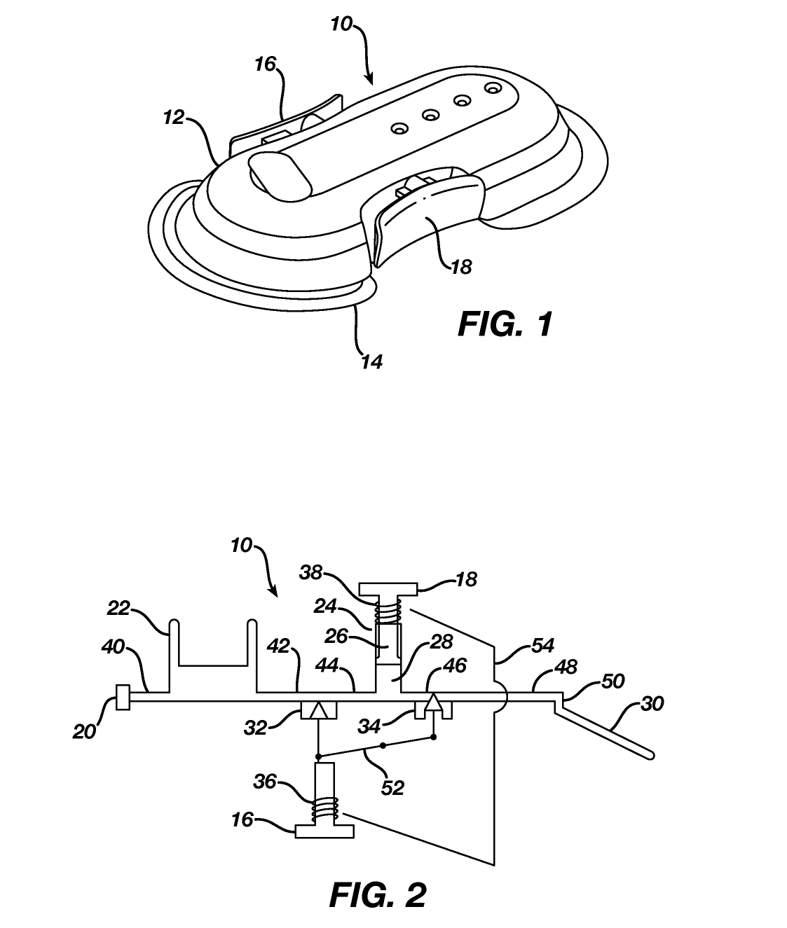

Referring now to FIG. 1 it is a perspective view of a first infusion device embodying certain aspects of the present invention. The device 10 generally includes an enclosure 12, a base 14, a first actuator control button 16, and a second actuator control button 18.

The enclosure 12, as will be seen subsequently, is formed by virtue of multiple device layers being brought together. Each layer defines various components of the device such as, for example, a reservoir, fluid conduits, pump chambers, and valve chambers, for example. This form of device construction, in accordance with aspects of the present invention, enables manufacturing economy to an extent rendering the device disposable after use.

The base 14 preferably includes an adhesive coating to permit the device to be adhered to a patient's skin. The adhesive coating may originally be covered with a releasable cover that may be pealed off of the base 14 when the patient endeavors to deploy the device 10. Such arrangements are well known in the art.

The device 10 may be mated with a previously deployed cannula assembly. However, it is contemplated herein that the various aspects of the present invention may be realized within a device that may be alternatively first adhered to the patient's skin followed by the deployment of a cannula thereafter.

The actuator buttons 16 and 18 are placed on opposites sides of the device 10 and directly across from each other. This renders more convenient the concurrent depression of the buttons when the patient wishes to receive a dose of the liquid medicament contained within the device 10. This arrangement also imposes substantially equal and opposite forces on the device during dosage delivery to prevent the device from being displaced and possibly stripped from the patient. As will be further seen hereinafter, the concurrent depression of the buttons is used to particular advantage. More specifically, the actuator button 16 may serve as a valve control which, when in a first position as shown, establishes a first fluid path between the device reservoir and the device pump to support pump filling, and then, when in a second or depressed position, establishes a second fluid path between the device pump and the device outlet or cannula to permit dosage delivery to the patient. As will be further seen, a linkage between the control actuator buttons 16 and 18 permits actuation of the device pump with the actuator control button 18 only when the second fluid path has been established by the first actuator control button 16. Hence, the first actuator control button 16 may be considered a safety control.

Referring now to FIG. 2, it is a schematic representation of the valves and pump of the device 10 of FIG. 1. As may be seen in FIG. 2, the device 10 further includes a fill port 20, a reservoir 22, a pump 24, and the cannula 30. The device further includes a first valve 32 and a second valve 34. Fluid conduit 40 provides a fluid connection between the fill port 20 and the reservoir 22, fluid conduit 42 provides a fluid connection between the reservoir 22 and the first valve 32, fluid conduit 44 provides a fluid connection between the first valve 32 and the pump 24, fluid conduit 46 provides a fluid connection between the pump 24 and the second valve 34, and fluid conduit 48 provides a fluid connection between the second valve 34 and the device outlet 50. The outlet 50 is arranged to communicate with the cannula 30.

It may also be noted that the actuator buttons 16 and 18 are spring loaded by springs 36 and 38. The springs are provided for returning the actuator buttons to the first position after a dosage is administered.

The pump 24 of the device 10 comprises a piston pump. The pump 24 includes a pump piston 26 and a pump chamber 28. In accordance with this embodiment, the actuator control button 18 is directly coupled to and is an extension of the pump piston 26.

With further reference to FIG. 2, the device additionally includes a first linkage 52 and a second linkage 54. The first linkage is a toggle linkage between the first valve 32 and the second valve 34. It is arranged to assure that the second valve 34 does not open until after the first valve 32 is closed. The second linkage 54 is between the first actuator button 16 and the second actuator button 18. It is arranged to assure that the pump does not pump until after the first valve is closed and the second valve is opened by the first actuator button 16.

Still further, the second valve 34 is a safety valve that closes tighter responsive to increased fluid pressure within fluid conduit 46. This assures that the liquid medicament is not accidentally administered to the patient notwithstanding the inadvertent application of pressure to the reservoir, for example. In applications such as this, it is not uncommon for the reservoir to be formed of flexible material. While this has its advantages, it does present the risk that the reservoir may be accidentally squeezed as it is worn. Because the second valve only closes tighter under such conditions, it is assured that increased accidental reservoir pressure will not cause the fluid medicament to flow to the cannula.

In operation, the reservoir is first filled through the fill port 20 to a desired level of medicament. In this state, the valves 32 and 34 will be as shown. The first valve 32 will be open and the second valve 34 will be closed. This permits the piston chamber 28 to be filled after the reservoir is filled. The cannula 30 may then be deployed followed by the deployment of the device 10. In this state, the valves 32 and 34 will still be as shown. The first valve 32 will be open and the second valve 34 will be closed. This permits the pump chamber 28 to be filled through a first fluid path including conduits 42 and 44 as the piston 24 returns to its first position after each applied dose.

When the patient wishes to receive a dose of medicament, the actuator buttons are concurrently pressed. In accordance with aspects of the present invention, the linkage 52 causes the first valve 32 to close and the second valve 34 to thereafter open. Meanwhile, the second linkage 54 precludes actuation of the pump 24 until the first valve 32 is closed and the second valve 34 is opened by the first actuator button 16. At this point a second fluid path is established from the pump 24 to the cannula 30 through fluid conduits 46 and 48 and the outlet 50. The medicament is then administered to the patient through cannula 30.

Once the medication dosage is administered, the piston 24, and thus the actuator button 18, is returned under the spring pressure of spring 38 to its initial position. During the travel of the piston back to its first position, a given volume of the liquid medicament for the next dosage delivery is drawn from the reservoir into the pump chamber 28 to ready the device for its next dosage delivery.

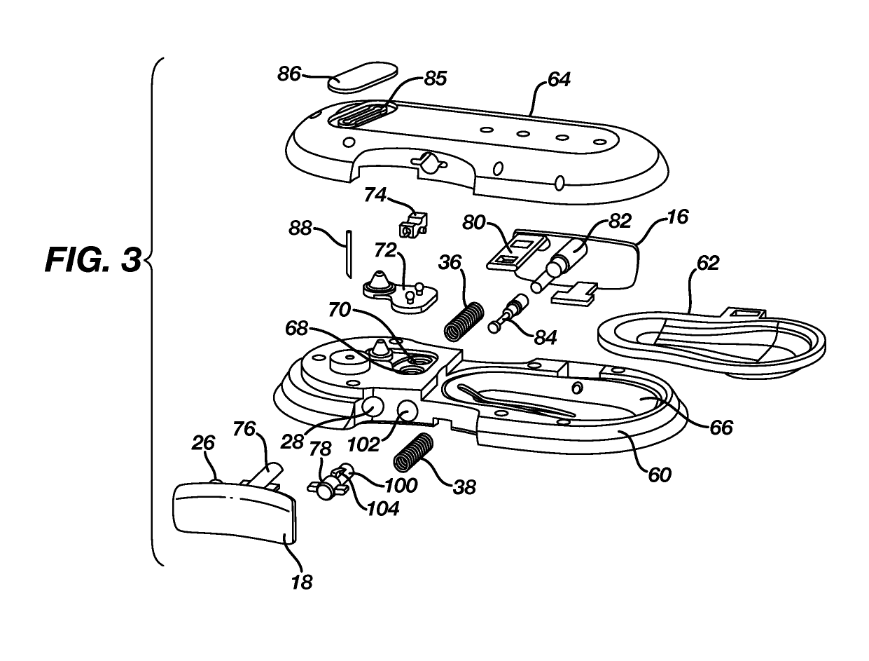

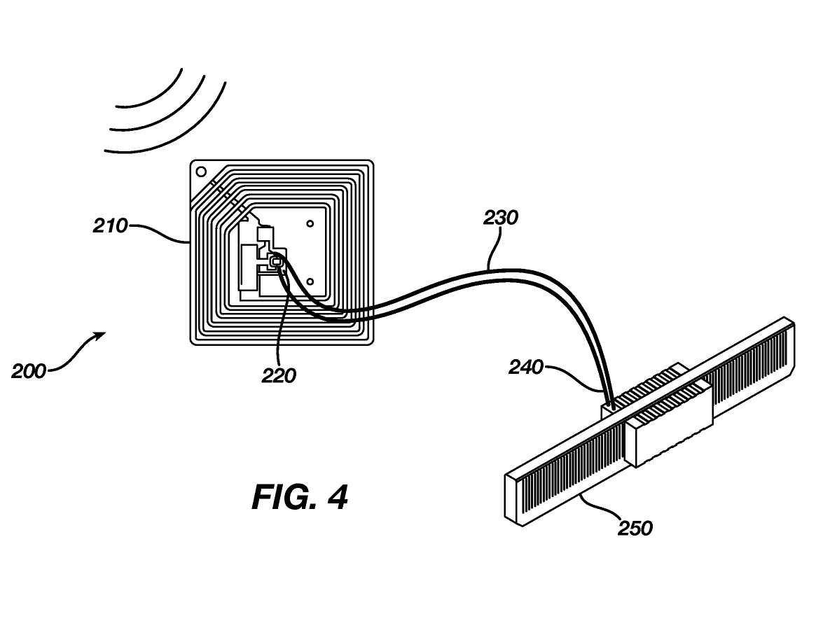

Referring now to FIG. 3, it is an exploded perspective view of the device of FIG. 1. It shows the various component parts of the device. The main component parts include the aforementioned device layers including the base layer 60, the reservoir membrane or intermediate layer 62, and the top body layer 64. The base layer is a substantially rigid unitary structure that defines a first reservoir portion 66, the pump chamber 28, and valve sockets 68 and 70 of the first and second valves respectively. The base layer 60 may be formed of plastic, for example. The reservoir membrane layer 62 is received over the reservoir portion 66 to form the reservoir 22 (FIG. 2). A valve seat structure 72 is received over the valve sockets 68 and 70 to form the first and second valves 32 and 34 (FIG.2) respectively. A rocker 74 is placed over the valves seat structure 72 to open and close the valves as will be seen subsequently. The pump actuator button 18 carries the pump piston that is received within the pump chamber 28. The pump actuator button 18 also carries a cam cylinder 76 with a lock tube 78 therein that form a portion of the second linkage 54 (FIG. 2). The spring 38 returns the actuator button 18 to its first position after each dosage delivery.

The first actuator control button carries a valve timing cam 80 that rocks the rocker 72. The button 16 further carries a cam cylinder 82 and a cam pin 84 that is received into the cam cylinder 82. The spring 36 returns the actuator button 16 to its first position after each dosage delivery. The top body layer 64 forms the top portion of the device enclosure. It receives a planar cap 86 that completes fluid paths 85 partially formed in the top layer 64. Lastly, a needle 88 is provided that provides fluid coupling from the cannula (not shown) to the outlet of the device 10.

The infusion system described herein is capable of delivering discrete doses of medication to the patient with each actuation of the buttons 16 and 18. Most, if not all, patients may desire a way for their infusion device to record when a dose is delivered. Historical information indicating when a patient received a dose is important in managing chronic conditions and diseases, such as diabetes. Insulin-dependent diabetics, for example, need to know how much insulin they have injected into their body and when, so that they can determine how much insulin they should receive to compensate for meals, etc.

It has been found that transmitting the occurrence of each dose to a remote device is desirable, as the structure and method for doing so minimizes the number of components that need to be added to the infusion device of FIGS. 1-3. A near-field communication (NFC) system, for example, can be used to transmit the occurrence of each dose a short distance. The power supply for this type of transmission system can be in the receiving device, rather than the transmitter which, in this example, is the infusion device. By eliminating the need for a power supply within the infusion device, the weight and size of the device is kept to a minimum, and the shelf-life of the device is not affected by the inclusion of a battery that can discharge over time or require specific storage conditions (temperature, etc.)

FIG. 4 illustrates an exemplary near-field transmission system 200 that may be added to the presently described infusion device as a means for counting and tracking dosing information via a remote device. The near-field communications (NFC) antenna 210 and associated near-field integrated circuit 220 are inexpensive and highly miniaturized. An inexpensive position encoder is also added to the pump. In this embodiment, the position encoder comprises a moving portion 250 and a stationary portion 240. The encoder is set to the fully-retracted position when the pump is completely full of medicament. Each time a dose is delivered by the infusion device, the moving portion 250 of the encoder moves relative to the stationary portion 240 of the encoder.

When the near-field transmission system is placed in proximity to a near-field receiver (not shown), the power necessary to operate the near field integrated circuit 220 is supplied by the receiver via inductive coupling between the receiver and the near-field antenna. While not wishing to be bound by theory, the receiver generates a magnetic field. When the near field antenna 210 is placed within the magnetic field, the magnetic field around the receiver creates a current within the near-field antenna 210, according to the principle of induction, thereby generating the electricity in the near-field transmitter system 200 to power the near-field integrated circuit 22. This obviates the need for a power supply to be placed within the infusion device.

The position of the moving portion 250 of the encoder relative to the stationary portion of the encoder 240 is then transmitted via electrical contacts 230 to the near-field integrated circuit 220. The near-field integrated circuit 220 processes the signal received via the electrical contacts 230 and the processed signal is transmitted to the remote device via the near-field antenna 210.

In the embodiment of the near-field transmitter system 200 of FIG. 3, the moving portion 250 of the position encoder may be mechanically linked to the piston actuator button 18 of FIG. 1 of an infusion device. Thus, each time the piston is actuator to deliver a bolus of medication, the moving portion 250 of the position encoder is advanced relative to the stationary portion 240 of the position encoder. The signal provided to a receiver will reflect the change in position and software running on the receiver can interpret the changed signal as an indication that a bolus was delivered. If the near-field transmitter system 200 is within the magnetic field produced by the receiver at the time of the piston's actuation, then the receiver may also record the time of the delivery and thus be able to maintain a chronological log of medication deliveries.

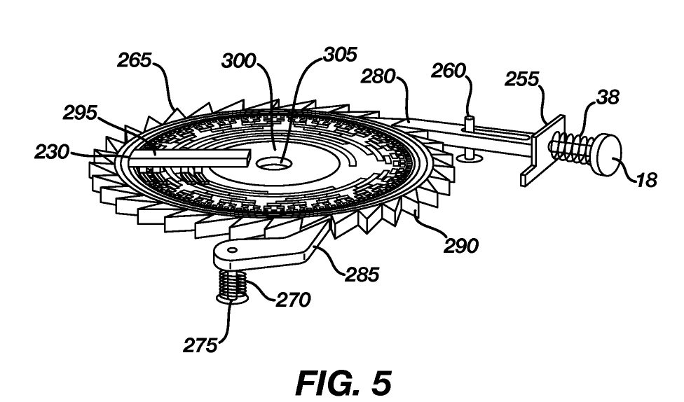

As illustrated in FIG. 5, the encoder may also be implemented as a rotary disk. When the user presses the pump's actuation button 18 to deliver medicament, a pawl-and-ratchet mechanism rotates the encoder disk 300 by one increment. The encoder disk 300 should include means to rotatably secure it within the housing. As illustrated, the encoder disk has a hole 305 to receive a mounting post or other structure known in the art to permit the encoder disk 300 to be held in place while still permitting rotation. Typically, the top cover of the infusion device will inhibit any vertical motion of the encoder disk 300 and the mounting structure need only retain the disk from horizontal movement.

The encoder disk may also have a series of teeth 290 disposed about its circumference and electrical contacts 265 on its exposed side. An encoder pickup 295 is disposed in proximity to the electrical contacts 265, but remains fixed relative to the housing of the infusion device, allowing the encoder disk 300 to move relative to the encoder pickup 295. The encoder disk 300 has electrical contacts 265 imprinted upon its surface. The electrical contacts 265 are arranged such that they open and close electrical circuits with the N electrical pickup contacts that ride on the surface of the encoder disk. The electrical contacts 265 provide a unique binary code for each discrete position of the encoder disk 300. With N electrical contacts, there are 2^{N}−1 possible unique binary codes. For example, if the infusion had the capability of being filled with 300 U of insulin and each actuation of the infusion device causes a discrete delivery of 1 U of insulin, the encoder disk must have at least 300 unique positions and 10 (i.e., 1+FLOOR[log₂(300)+1]) electrical contacts to detect each possible state of the pump.

NFC technology is often used to identify or prevent counterfeits. The NFC ID in the pump could be programmed with a secure code and encryption scheme that would be unknown to counterfeiters. While this would not prevent the manufacture or use of a counterfeit pump, such a pump could be detected and recognized through encrypted NFC communications.

The IC could also be factory-programmed with information such as pump date-of-manufacture, batch code, and model number. The pump manufacturer could use this information for inventory control and in forensic investigations.

The ratchet-and-pawl system shown in FIG. 5 may include the pawl 255 in mechanical contact with the piston actuation button 18 and spring 38. The pawl may include an opening for the limiter 260 to be placed such that it limits the movement of the pawl 255 to ensure that the encoder disk 300 is moved only a single increment with each actuation, by the pawl tip 280 biasing against the teeth 290 of the encoder disk 300 when the piston actuation button 18 is depressed. The ratchet ensure that the encoder disk can only rotate in a single direction and may include a ratchet post 275 with a ratchet spring 270 and ratchet arm 285. The ratchet arm is biased against the teeth 290 of the encoder gear 300 by the ratchet spring 270. The ratchet spring is attached to or otherwise engaged with the ratchet post 275 for the ratchet spring to bias against.

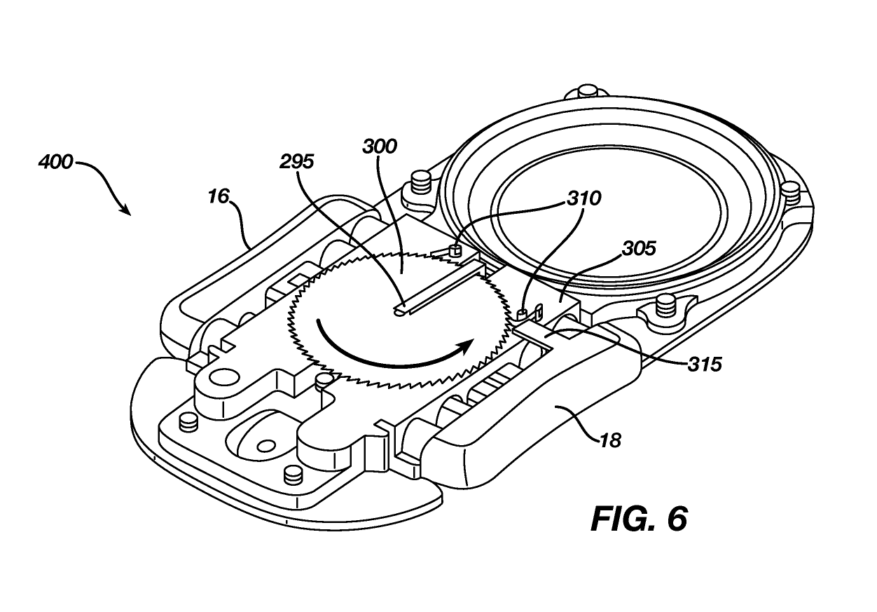

FIG. 6 illustrates a manually-activated, mechanical infusion device 400. Such devices are also described in commonly-assigned U.S. Pat. No. 7,976,500, which is hereby incorporated by reference in its entirety. The encoder disk 300 is rotatably mounted on a top layer 7 of the pump mechanism. An encoder pickup 295 is fixedly mounted over the encoder disk 300, so that the encoder pickup 295 is able to sense the state of the electrical contacts 265 on the encoder disk 300. In this embodiment, the actuator button 16 is depressed to permit motion of the actuator button 18. When actuator button 18 is depressed, a delivery of medication occurs. As well, the pawl 315 biases pawl mechanism 305, which is limited in movement by the pawl stop post 260. A ratchet mechanism 310 ensures that the encoder disk 300 cannot rotate in a counter-clockwise direction. The ratchet mechanism may have similar or identical structure to that shown in FIG. 5.

The receiver that supplies power for and receives data from the near field transmitter system of the disclosed embodiments can be a cell phone or other device equipped with a near field receiver. Software on the receiver device may perform numerous functions, such as logging the time of each dose delivered by the pump or to determine the amount of medication remaining in the pump at a given time.

The system may determine the amount of medicament in the pump in accordance with the following illustrative example. The infusion pump's encoder and IC remain unpowered until the user wants to determine the medicament remaining in the infusion pump. The user then positions an NFC equipped device, such as a cell phone, within a few centimeters of the infusion pump. The infusion pump's NFC antenna receives enough electromagnetic energy from the cell phone's NFC transmitter to power the infusion pump's IC and the encoder. The IC reads the encoder's position and wirelessly transmits it to the cell phone, where it will be available for display, recording and further processing.

The position encoder could be built using a variety of technologies known in the art including resistive, magnetic, LVDT, binary conductive, capacitive, inductive and optical. Encoder technologies could be combined to achieve the best combination of cost, durability, reliability, accuracy and resolution.

While particular embodiments of the present invention have been shown and described, modifications may be made. For example, instead of manual actuation and spring loaded return of the valves used herein, constructions are possible which perform in a reversed manner by being spring actuated and manually returned. It is therefore intended in the appended claims to cover all such changes and modifications which fall within the true spirit and scope of the invention as defined by those claims.

What is claimed is:

1. A wearable infusion device comprising:
a reservoir that holds a liquid medicament;
an outlet port that delivers the liquid medicament to a patient;
a pump that displaces a volume of the liquid medicament when actuated;
at least a first valve, a second valve, and a third valve, the valves establishing fluid connection between the reservoir and the pump when in a first valve configuration and establishing fluid connection between the pump and the outlet when in a second valve configuration, wherein at least two of the valves are closed to prevent fluid flow from the reservoir to the outlet when the valves are in the first valve configuration, wherein the first, second, and third valves comprise a shuttle valve;
an encoder disk rotatably mounted in the infusion device;
an encoder pickup fixedly mounted in proximity in the infusion device; and
a near-field antenna in electrical communication with the encoder pickup;
wherein the encoder disk is configured to move at least one discrete increment when the pump is actuated.

2. The device of claim 1, comprising a first control that sets the configuration of the valves and a second control that actuates the pump.

3. The device of claim 2, further comprising a linkage that precludes the second control from actuating the pump until the first control sets the valves into the second valve configuration.

4. The device of claim 3, wherein the valves are arranged to prevent the outlet from being in fluid connection with the reservoir.

5. The device of claim 1, wherein the valves are arranged to prevent the outlet from being in fluid connection with the reservoir.

6. The device of claim 1, comprising a first control that sets the configuration of the valves and a second control that actuates the pump.

7. The device of claim 6, further comprising a linkage that precludes the second control from actuating the pump until the first control sets the valves into the second valve configuration.

8. The device of claim 1, wherein the valves are operable independently from the pump.

9. The device of claim 1, wherein the valves and the pump are operable solely by manual force applied to the first and second control.

10. The device of claim 1, wherein the pump is a piston pump.

11. The device of claim 10 wherein the encoder disk comprises a plurality of teeth along its circumference.

12. The device of claim 11 wherein the encoder disk comprises a plurality of electrical contacts disposed on its surface.

13. The device of claim 12 wherein the number of teeth along the circumference of the encoder disk is proportional to the size of the reservoir.

14. The device of claim 12 wherein the number of electrical contacts is equal to or greater than the result of the formula 1+FLOOR[log2(N)+1], where N is the number of teeth disposed along the circumference of the encoder disk.

15. The device of claim 10 comprising a ratchet system to permit the encoder disk to rotate in a single direction.

16. The device of claim 10 comprising a pawl configured to bias against the teeth disposed along the circumference of the encoder wheel 300 when the pump is actuated.
